(19) United States
(12) Patent Application Publication
(10) Pub. No.: US 2025/0064888 A1
(43) Pub. Date: Feb. 27, 2025

(54) COMPOSITIONS AND METHODS FOR TREATING CYTOKINE STORM

(71) Applicant: SHENZHEN HIGHTIDE BIOPHARMACEUTICAL LTD., Shenzhen (CN)

(72) Inventors: Liping Liu, Shenzhen (CN); Ru Bai, Shenzhen (CN)

(21) Appl. No.: 18/727,078

(22) PCT Filed: Jan. 20, 2023
(86) PCT No.: PCT/CN2023/073344

§ 371 Date: Jul. 6, 2024

Related U.S. Application Data
(60) Provisional application No. 63/302,878, filed on Jan. 25, 2022.

(51) Int. Cl. A61K 38/10; A61K 9/00; A61K 45/06; A61P 29/00; A61P 31/14

(52) U.S. Cl. CPC A61K 38/10; A61K 9/0019; A61K 45/06; A61P 29/00; A61P 31/14

(57) ABSTRACT

The invention provides compositions and methods for treating, reducing or preventing cytokine storm, for example, in connection with a viral infection (e.g., by SARS-CoV-2), drug treatment or therapy, trauma or an inflammatory disease, or a related disease and disorder.

### PRIORITY CLAIMS AND RELATED PATENT APPLICATIONS

This application claims the benefit of priority to U.S. Provisional Application Ser. No. 63/302,878, filed Jan. 25, 2022, the entire content of which is incorporated herein by reference.

### TECHNICAL FIELD OF THE INVENTION

The invention generally relates to pharmaceuticals and therapeutic uses thereof. More particularly, the invention provides compositions and methods for reducing pro-inflammatory cytokines and treating cytokine storm, for example, associated with viral infections (e.g., SARS-CoV-2 or COVID-19), and related diseases and disorders.

### BACKGROUND OF THE INVENTION

COVID-19 is a contagious disease caused by severe acute respiratory syndrome coronavirus 2 (SARS-CoV-2). SARS-CoV-2 virus can spread from an infected person's mouth or nose in small liquid particles when they cough, sneeze, speak or breathe. These particles range from larger respiratory droplets to smaller aerosols. A virus surface spike protein mediates SARS-CoV-2's entry into human cells.

Symptoms of COVID-19 are variable, but often include fever, cough, fatigue, breathing difficulties, and loss of smell and taste. While most people have mild symptoms, a significant percentage of people develop acute respiratory distress syndrome (ARDS). ARDS can be precipitated by cytokine storms, multi-organ failure, septic shock, and blood clots. Longer-term damage to organs, in particular the lungs and heart, has been frequently observed. (See, e.g., Ye, et al. 2020 The Journal of Infection 80 (6): 607-613; Brevini, T., Maes, M., Webb, G. J, et al. Nature 2022.)

Although COVID-19 patients at the early onset may experience mild symptoms, the conditions can suddenly deteriorate at a later stage of disease progression. One possible cause is an inflammatory storm that is frequently initiated at the later stage. This inflammatory storm can lead to failure of various organ functions, and some patients die due to non-pulmonary multiple organ failure. (Li, et al. 2020) Journal of Pharmaceutical Analysis 10 (2): 102-108.)

Increasing clinical evidence has shown that some patients experience long-term effects from their infection, known as post-COVID conditions (a.k.a., post-acute COVID-19, long COVID or chronic COVID, etc.). These patients experience returning or ongoing health problems long after (e.g., weeks, months or years after) being infected COVID-19. It has been reported that between one month and one year after having COVID-19, 1 in 5 people ages 18 to 64 has at least one medical condition that may be due to COVID-19 infection. (See, e.g., Perlis, et al. 2022 JAMA Netw Open. 5 (10); Household Pulse Survey, National Center for Health Statistics, 2022 https://www.cdc.gov/nchs/covid19/pulse/long-covid.htm; Levine, et al. “Long COVID Research, Services, And Supports: A Call To Action” Aug. 2, 2022 Health Affairs Forefront 10.1377/forefront.20220801.50344.)

Symptoms of post-COVID conditions can be wide ranging, including fatigue, fever, respiratory and heart symptoms (e.g., difficulty breathing or shortness of breath, cough, chest pain, heart palpitations), neurological symptoms (e.g., difficulty thinking or concentrating, sometimes referred to as “brain fog”), headache, sleep problems, lightheadedness, depression or anxiety, digestive symptoms (e.g., diarrhea, stomach pain), joint or muscle pain, rash, etc. (See, e.g., Cassar, et al. 2021 EClinicalMedicine 41, 101159; Singh, et al. 2020 Am J Physiol Cell Physiol 319: C258-C267; Ajaz, et al. 2020 Am J Physiol Cell Physiol 320: C57-C65, 2021; Gibellini, et al. 2020 EMBO Molecular Medicine 12: e13001; Raman, et al. 2022 Euro. Heart J. 43, 1157-1172; Evans, et al. 2022 Lancet Respir. Med. 10:761-75; Singh, et al. 2022 Chest 161(1):54-63; Shields, et al. 2022 Clin. and Exper. Immun. 209, 3:247-258; Pretorius, et al. 2021 Cardio Diabetology 20, No. 172.)

The term “cytokine storm”, first described in the medical literature in 1993, is a severe form of systemic inflammatory response triggered by various factors such as infection or certain drugs. An experimental study of hematological malignancies model was reported by Alegre et al. in 1991 in which a similar term “cytokine release syndrome” (CRS) was used. (Ferrara, et al. 1993 Transplant Proc. 25 (1 pt 2): 1216-7; Alegre, et al. 1991 J Immunol. 146 (4): 1184-91.) Since then, studies related to cytokine storm have been reported in connection with different viral diseases, bacterial infections, hemophagocytic lymphohistiocytosis, multiple sclerosis, pancreatitis, and other inflammatory diseases, which can cause cytokine storm and lead to multiple organ dysfunction syndrome (MODS). The most severely affected organs are usually the lung, cardiovascular system and kidney. (Karlsson, et al. 2007 IntensiveCare Med. 33 (3): 435-43.)

In a cytokine storm, inflammatory cytokines continue to be produced in large quantities after the body is infected with viruses or microorganisms, as well as when the body's immune function is abnormal, leading to continued activation of more immune cells to accumulate to the inflammatory site. A variety of cytokines in body fluids (e.g., TNF-α, IL-1, IL-6, IL-12, IFN-α, IFN-β, IFN-γ, MCP-1 and IL-8) are rapidly produced in large quantities. (Huang, et al. 2020 Lancet 395 (10223): 497-506.) Excessive immune cells and a variety of inflammatory cytokines can cause tissue congestion, edema, fever and injury, which are important causes of acute respiratory distress syndrome. They may also cause other secondary infections or even lead to systemic inflammatory response syndrome. It has been reported that severely ill COVID-19 patients usually show a significant increase in inflammatory cytokines such as IL-6, TNF-α, and IFN-γ, which are characteristics of a cytokine storm. Intense immune cytokine storm occurs in severely ill patients with COVID-19 when immune cells attack their own normal tissues. Cytokine storms bring great challenges to clinical care for the severely ill COVID-19 patients who face a high death rate during multiple attacks.

Currently, prevention and treatment of COVID-19 include vaccination, supportive therapy, symptomatic treatment, and antiviral agents. Various antiviral medications have been investigated for COVID-19 with some showing efficacy and reduction of mortality. In November 2020, the U.S. FDA issued an EUA for the investigational monoclonal antibody therapy bamlanivimab for the treatment of mild-to-moderate COVID-19 and also granted EUA for casirivimab and imdevimab to be administered together for the treatment of mild to moderate COVID-19. In December 2021, emergency use authorizations (EUA) were granted in the U.S. for antiviral medications PAXLOVID™ (nirmatrelvir; ritonavir), developed by Pfizer, and LAGEVRIO™ (molnupiravir), developed by Merck.

Despite these encouraging developments, vaccine-resistant and/or treatment-resistant viral variants have continued to emerge as the pandemic spreads to more vulnerable demographic populations including immunocompromised patients and high-risk groups such as seniors and others who do not response well to vaccines or currently available treatments.

Therapeutics to antagonize the function of inflammatory cytokines to reduce the production of inflammatory cytokines have been studied. The purpose of antagonizing the function of inflammatory cytokines and down-regulating the inflammatory response can be achieved by using monoclonal antibodies against IL-1, TNF-α, IL-6, IL-8, etc., or their receptor antagonists. Clinical trials, however, have shown that such immunomodulatory therapies have poor effects in the treatment of inflammatory storm and cannot significantly reduce the morbidity and mortality. The potential explanations may be multifaceted: (1) when antagonizing the harmful effects of cytokines, they also weaken their normal physiological functions; (2) cytokine storm is a complex combination of multiple inflammatory factors so that the effect of using a single immune agent is not noticeable; (3) there are different subtypes of cytokine receptors, and their functions may also be different; and (4) there is an increased risk of the occurrence of compensatory anti-inflammatory response syndrome.

Currently available therapeutics and methods for reducing pro-inflammatory cytokines and treating cytokine storm remain inadequate. In particular, there is an urgent unmet need for a safe and effective treatment for cytokine storm associated with viral infections such as by COVID-19.

### SUMMARY OF THE INVENTION

The invention is based in part on the surprising discovery that certain Reg3α analogs can be effectively used to reduce pro-inflammatory cytokines and/or to treat, reduce or prevent cytokine storm, for example, in connection with a viral infection (e.g., by COVID-19), drug treatment or therapy (e.g., during CAR-T therapy), trauma or an inflammatory disease.

Regenerating islet-derived (Reg) protein, as a stress protein secreted by the body during an inflammatory response, can play a key role in prognosis of certain diseases, for example, through anti-inflammation, anti-apoptosis/necrosis, and/or antibacterial effect to prevent complications, as well as promotion of cell proliferation and tissue neogenesis. The rapid increase of Reg protein levels to a higher level at the early stage in the disease onset is important in controlling the development of and recovery from the disease. The up-regulation of endogenous Reg protein, however, has a lag of about 48-72 hours. In addition, with the increase in age or disease comorbidities, the increase rate of Reg protein expression level can be significantly reduced. Due to the lack of Reg protein protection, the corresponding delayed inflammatory response, aggravated bacterial infiltration, and higher C-reactive protein level may occur. The loss of Reg protein protection diminishes the patient's ability to resist tissue or organ damage caused by the disease. Lacking effective protection of Reg protein thus can lead to inflammatory response and injury to organs and tissues.

A stable and effective analogue of the active center of Reg3α is disclosed herein. Without wishing to be bound by the theory, it is believed that the disclosed compound can significantly reduce or alleviate the severity of body inflammation and play a beneficial role in reducing or preventing severe systemic inflammatory response or cytokine storm in COVID-19 patients. The levels of inflammatory cytokines TNF-α, IL-6 and IL-1β, which are the most important inflammatory cytokines involved in the inflammatory response, can be significantly reduced. The disclosed compound can significantly decrease the infiltration of neutrophils and macrophages and significantly lessen the severity of injury (including apoptosis and necrosis), thus helping with preventing or lessening complications and promoting recovery, which are of great significance in controlling the disease and improving prognosis.

In one aspect, the invention generally relates to a method for treating SARS-CoV-2 infection, or a related disease or condition, comprising administering to a subject in need thereof a pharmaceutical composition comprising a peptide which is an analogue of the active center of Reg3α.

In another aspect, the invention generally relates to a method of reducing pro-inflammatory cytokines, the method comprising administering to a subject in need thereof a pharmaceutical composition comprising a peptide which is an analogue of the active center of Reg3α.

In yet another aspect, the invention generally relates to a method of treating hypercytokinemia or cytokine storm or a disease or condition involving hypercytokinemia or cytokine storm, the method comprising administering to a subject in need thereof a pharmaceutical composition comprising a peptide which is an analogue of the active center of Reg3α.

In certain embodiments, the amino acid sequence of the active center of Reg3α is H-IGLHDPSHGTLPNGS-OH.

In certain embodiments, the analogue of the active center of Reg3α is Ac-IGLHDPSHGTLPAGS, or a pharmaceutically acceptable form thereof.

In yet another aspect, the invention generally relates to a pharmaceutical composition comprising Ac-IGLHDPSHGTLPAGS, or a pharmaceutically acceptable form thereof, and a pharmaceutically acceptable excipient, carrier, or diluent, suitable for treating SARS-CoV-2 infection, or a related disease or condition.

In yet another aspect, the invention generally relates to a unit dosage form comprising a pharmaceutical composition disclosed herein.

In yet another aspect, the invention generally relates to use of Ac-IGLHDPSHGTLPAGS, or a pharmaceutically acceptable form thereof, for treating SARS-CoV-2 infection, or a related disease or condition.

In yet another aspect, the invention generally relates to use of Ac-IGLHDPSHGTLPAGS, or a pharmaceutically acceptable form thereof, for reducing pro-inflammatory cytokines, or treating hypercytokinemia or cytokine storm or a disease or condition involving hypercytokinemia.

In yet another aspect, the invention generally relates to use of Ac-IGLHDPSHGTLPAGS, or a pharmaceutically acceptable form thereof, for preventing or treating post-COVID conditions, or a related disease or disorder.

In yet another aspect, the invention generally relates to use of Ac-IGLHDPSHGTLPAGS, or a pharmaceutically acceptable form thereof, for the manufacture of a medicament for preventing or treating post-COVID conditions, or a related disease or disorder.

### BRIEF DESCRIPTION OF THE DRAWINGS

FIG. 1 show exemplary data on effects of Ac-IGLHDPSHGTLPAGS on the survival rate in LPS induced mice model.

### DEFINITIONS

Unless defined otherwise, all technical and scientific terms used herein have the same meaning as commonly understood by one of ordinary skill in the art to which this invention belongs. The following terms, unless indicated otherwise according to the context wherein the terms are found, are intended to have the following meanings.

When trade names are used herein, the trade name includes the product formulation, the generic drug, and the active pharmaceutical ingredient(s) of the trade name product, unless otherwise indicated by context.

Ranges provided herein are understood to be shorthand for all of the values within the range. For example, a range of 1 to 14 is understood to include any number, combination of numbers, or sub-range from the group consisting 1, 2, 3, 4, 5, 6, 7, 8, 9, 10, 11, 12, 13 or 14.

As used herein, “at least” a specific value is understood to be that value and all values greater than that value.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

As used herein, the term “antibody” refers to molecules that are capable of binding an epitope or antigenic determinant. The term is meant to include whole antibodies and antigen-binding fragments thereof. The term encompasses polyclonal, monoclonal, chimeric, Fabs, Fvs, single-chain antibodies and single or multiple immunoglobulin variable chain or CDR domain designs as well as bispecific and multispecific antibodies. Antibodies can be from any animal origin. Preferably, the antibodies are mammalian, e.g., human, murine, rabbit, goat, guinea pig, camel, horse and the like, or other suitable animals. Antibodies may recognize polypeptide or polynucleotide antigens. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid antibody molecules, F(ab)₂ and F(ab) fragments; Fv molecules (for example, noncovalent heterodimers), dimeric and trimeric antibody fragment constructs; minibodies, humanized antibody molecules, and any functional fragments obtained from such molecules, wherein such fragments retain specific binding.

As used herein, the terms “disease”, “disorder” or “condition” are used interchangeably herein to refer to a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

As used herein, the term “modulate” refers to the production, either directly or indirectly, of an increase or a decrease, a stimulation, inhibition, interference, or blockage in a measured activity when compared to a suitable control. A “modulator” of a polypeptide or polynucleotide refers to a substance that affects, for example, increases, decreases, stimulates, inhibits, interferes with, or blocks a measured activity of the polypeptide or polynucleotide, when compared to a suitable control. For example, a “modulator” may bind to and/or activate or inhibit the target with measurable affinity, or directly or indirectly affect the normal regulation of a receptor activity.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the terms “prevent”, “preventing”, or “prevention” refer to a method for precluding, delaying, averting, or stopping the onset, incidence, severity, or recurrence of a disease or condition. For example, a method is considered to be a prevention if there is a reduction or delay in onset, incidence, severity, or recurrence of a disease or condition or one or more symptoms thereof in a subject susceptible to the disease or condition as compared to a subject not receiving the method. The disclosed method is also considered to be a prevention if there is a reduction or delay in onset, incidence, severity, or recurrence of one or more symptoms of a disease or condition in a subject susceptible to the disease or condition after receiving the method as compared to the subject's progression prior to receiving treatment. The reduction or delay in onset, incidence, severity, or recurrence of osteoporosis can be about a 5, 10, 20, 30, 40, 50, 60, 70, 80, 90, 100%, or any amount of reduction in between.

Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

As used herein, the terms “subject” and “patient” are used interchangeably herein to refer to a living animal (human or non-human). The subject may be a mammal. The terms “mammal” or “mammalian” refer to any animal within the taxonomic classification mammalia. A mammal may be a human or a non-human mammal, for example, dogs, cats, pigs, cows, sheep, goats, horses, rats, and mice. The term “subject” does not preclude individuals that are entirely normal with respect to a disease or condition, or normal in all respects.

As used herein, the terms “treatment” or “treating” a disease or disorder refers to a method of reducing, delaying or ameliorating such a condition, or one or more symptoms of such disease or condition, before or after it has occurred. Treatment may be directed at one or more effects or symptoms of a disease and/or the underlying pathology. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Any compositions or methods disclosed herein can be combined with one or more of any of the other compositions and methods provided herein.

### DETAILED DESCRIPTION OF THE INVENTION

The invention provides certain analogs of the active center of Reg3α and compositions thereof that can be effectively used to reduce pro-inflammatory cytokines and prevent or reduce cytokine storm in a viral infection (e.g., by COVID-19), drug treatment or therapy (e.g., during CAR-T therapy), trauma or an inflammatory disease.

Reg proteins are involved in the growth and differentiation of cells from various organs under normal and disease conditions. They were discovered in the 1970s and 1980s and was named regenerating protein because of its up-regulated expression after organ injury. It was thought to promote regeneration effects. (Terazono, et al. 1988 Journal of Biological Chemistry 263:2111-2114; Terazono, et al. 1990 Diabetologia. 33:250-252.) Subsequently, researchers from various fields named it pancreatitis-associated protein (PAP), pancreatic stone protein (PSP), and hepatocarcinoma-intestine-pancreas (HIP) according to their roles in various types of diseases. With the development of protein sequencing technology, these proteins are found to belong to the Reg protein family and were later collectively referred to as “Regenerating protein” or “Reg protein”. (Parikh, et al. 2012 Biomol Concepts. 3(1):57-70.) Reg protein is a self-secreted stress protein with important protective effects when the body is damaged (especially acute and severe body injury), and the expression of Reg protein is significantly up-regulated in various body inflammatory responses such as acute pancreatitis, septicemia (sepsis), peritonitis, and acute liver injury, and plays an important protective role in the development and prognosis of the diseases. (Reding, et al. 2017 Oncotarget. 8(18):30162-30174.)

As early as 1994, a clinical trial observed that Reg protein expression was significantly elevated in all patients on the first day of admission, reached the peak plasma concentration on days 2-4, and then gradually decreased to the basal level. It was observed that the rate of increase in the expression level of Reg protein was significantly slower for older patients than other patients, indicating that with the increase of age, the protection of Reg protein may be gradually lost, reducing the patient's ability to resist disease damage. In addition, the study also showed that the rapid increase of Reg protein level to a higher level in the early stage of disease was very important for the control of and recovery from the disease. (Iovanna, et al. 1994 Gastroenterology. 106(3):728-734.) These findings were later confirmed in other studies in different disease areas.

Regenerating islet-derived protein 3 alpha or Reg3α (a.k.a HIP/PAP (Hepatocarcinoma-Intestine-Pancreas/Pancreatitis-Associated Protein) is a protein that in humans is encoded by the REG3α gene. (Dusetti, et al. 1994 Genomics 19 (1): 108-14.) This gene encodes a pancreatic secretory protein that may be involved in cell proliferation or differentiation. The enhanced expression of this gene was observed during pancreatic inflammation and liver carcinogenesis.

Boeck et al. evaluated the role of Reg protein in ventilator-associated pneumonia (VAP) by retrospectively analyzing Reg protein levels in frozen serum samples from onset to day 7 in 101 patients clinically diagnosed with VAP, so as to assess the correlation between the Reg protein levels and the organ failure and prognosis, with the primary endpoint of death within 28 days after VAP onset. The results of this study showed that Reg protein level had a good correlation with the prognosis of patients and could be used as a biomarker related to organ failure and prognosis in patients with this type of pneumonia. (Boeck, et al. 2011 Chest. 140(4):925-932.) Scherr et al. also observed significantly increased Reg protein levels in 200 patients with chronic obstructive pulmonary disease (COPD) caused by bacterial infection, which has a direct correlation with disease severity and mortality. (Scherr, et al. 2013 Chest. 143(2):379-387.) Qian et al. evaluated the therapeutic effect of adipose-derived stem cells (ADSCs) on acute lung injury induced by Staphylococcus aureus in mice using ADSCs. The experimental results showed that intratracheal injection of ADSCs could reduce the severity of pulmonary inflammation, and reduce bacterial load and mortality in infected mice. In addition, experiments have demonstrated that ADSCs play a protective and antibacterial role against lung injury through Reg protein, and further studies have shown that the secretion of Reg protein is mediated through the TLR2-MyD88-JAK2/STAT3 pathway. (Qian, et al. 2016 Stem Cells 34(7):1947-56.)

A recent study was conducted to test whether a Reg3α analog could protect pancreatic acinar cell necrosis and reduce the inflammatory response of acute pancreatitis. The results showed that the Reg3α analog could reduce the severity of acute pancreatitis and had a significant effect in inhibiting systemic inflammatory response, in which the levels of pro-inflammatory cytokines TNF-α, IL-6 and IL-1β were significantly reduced. The Reg3α analog significantly reduced the infiltration of neutrophils and macrophages and the severity of injury (including apoptosis and necrosis). A further study found that the Reg3α analog could down-regulate the expression of Toll-like receptor 4 (TLR4) protein, and TLR4 gene knockout in mice made the beneficial protective effect of the Reg3α analog unable to be exerted. This demonstrated that the Reg3α analog alleviates the severity of body inflammation through TLR4 signaling pathway, and further plays a significant protective role. (Wu, et al. 2019. Biochem Biophys Res Commun. 512 (4): 670-677.)

Coronavirus attaches to host cells via the trimeric spike S glycoprotein. TLR4 of host cells recognizes S proteins and leads to the activation of inflammatory cytokines through a MyD88-dependent signaling pathway. The interaction between virus and cells leads to the massive production of immune mediators. In response to viral infection, infected cells promote the secretion of a large number of inflammatory cytokines (IL-1, IL-6, IL-8, IL-21). These cytokines, in turn, recruit lymphocytes and leukocytes to the site of infection.

In one aspect, the invention generally relates to a method of reducing pro-inflammatory cytokines, the method comprising administering to a subject in need thereof a pharmaceutical composition comprising a peptide which is an analogue of the active center of Reg3α.

In yet another aspect, the invention generally relates to a method for treating SARS-CoV-2 infection, or a related disease or condition, comprising administering to a subject in need thereof a pharmaceutical composition comprising a compound that is a peptide which is an analogue of the active center of Reg3α.

In yet another aspect, the invention generally relates to a method of treating, reducing or preventing hypercytokinemia or cytokine storm or a disease or condition involving hypercytokinemia or cytokine storm, the method comprising administering to a subject in need thereof a pharmaceutical composition comprising a peptide which is an analogue of the active center of Reg3α.

In yet another aspect, the invention generally relates to a method of treating, reducing or preventing an inflammatory disease or condition involving acute respiratory system or lung failure, the method comprising administering to a subject in need thereof a pharmaceutical composition comprising a peptide which is an analogue of the active center of Reg3α.

In yet another aspect, the invention generally relates to a method of treating, reducing or preventing an inflammatory disease or condition involving acute liver failure, the method comprising administering to a subject in need thereof a pharmaceutical composition comprising a peptide which is an analogue of the active center of Reg3α.

In certain embodiments, the amino sequence of the active center of Reg3α is Ac-H-IGLHDPSHGTLPNGS-OH.

In certain embodiments, the peptide which is an analogue of the active center of Reg3α is Ac-IGLHDPSHGTLPAGS or a pharmaceutically acceptable form thereof.

In certain embodiments, the subject is suffering from an overproduction of immune cells and/or pro-inflammatory cytokines into the lungs of the subject.

In certain embodiments, the subject is suffering from an overproduction of immune cells and/or pro-inflammatory cytokines into the liver of the subject.

In certain embodiments, the pro-inflammatory cytokines comprise one or more of TNF-α, IL-1, IL-6, IL-12, IFN-α, IFN-β, IFN-γ, MCP-1, IL-8, IL-2, IL-7, IL-18, IL-17, CCL-2, IP-10, MCP-3 and GM-CSF.

In certain embodiments, the subject is in a mild or moderate condition of SARS-CoV-2 infection, or a related disease or condition. In certain embodiments, the subject is in a severe or critical condition of SARS-CoV-2 infection, or a related disease or condition.

In certain embodiments, the subject suffers a post-COVID condition.

In certain embodiments, the related disease or condition is pneumonia and/or lung injury. In certain embodiments, the related disease or condition is acute respiratory distress syndrome. In certain embodiments, the related disease or condition is systemic inflammatory response syndrome.

In certain embodiments, the inflammatory disease or condition is hepatitis. In certain embodiments, the inflammatory disease or condition is severe hepatitis.

In certain embodiments, the inflammatory disease or condition is pneumonia.

In certain embodiments, the disease or condition being treated comprises an inflammatory condition.

In certain embodiments, the disease or condition being treated comprises acute respiratory distress syndrome.

In certain embodiments, the disease or condition is a cardiovascular condition.

In certain embodiments, the related disease or condition is failure of one or more organs.

In certain embodiments, at least one of the organs is selected from lung and liver.

In certain embodiments, the disease or condition being treated is an infection. In certain embodiments, the infection is a viral, bacterial, fungal or parasitic infection. In certain embodiments, the viral infection is caused by a coronavirus. In certain embodiments, the coronavirus is SARS-CoV-2.

In certain embodiments, at least 60% of severe SARS-CoV-2 patients with elevated inflammatory factors see their IL-6 reduced to normal within 7 days of treatment. In certain embodiments, at least 75% of severe SARS-CoV-2 patients with elevated inflammatory factors see their IL-6 reduced to normal within 7 days of treatment. In certain embodiments, at least 85% of severe SARS-CoV-2 patients with elevated inflammatory factors see their IL-6 reduced to normal within 7 days of treatment. In certain embodiments, at least 95% of severe SARS-CoV-2 patients with elevated inflammatory factors see their IL-6 reduced to normal within 7 days of treatment.

In certain embodiments, the administration of the compound results in prevention or reduction of SARS-CoV-2 patients from developing hypercytokinemia.

In certain embodiments, the disease or condition is due to a drug treatment or therapy (e.g., is CAR-T therapy).

In certain embodiments, the disease or condition is trauma.

In certain embodiments, administration of the analogue of the active center of Reg3α is via subcutaneous administration. In certain embodiments, administration of the analogue of the active center of Reg3α is via intravenous administration. In certain embodiments, administration of the analogue of the active center of Reg3α is via intramuscular administration. In certain embodiments, administration of the analogue of the active center of Reg3α is via inhaled administration.

The daily dose can be administered in a single dose or in two or more doses according to the particular circumstances.

In certain embodiments, the first dose is administered after the onset of hypercytokinemia or cytokine storm.

In certain embodiments, the first dose is administered in about 5 min to about 1 day (e.g., about 5 min to about 12 h, about 5 min to about 3 h, about 1 min to about 1 h.) after the onset of hypercytokinemia or cytokine storm.

The dosage may be administered in the range equivalent to about 2.5 to about 25 mg/kg/day (e.g., about 2.5 to 15 mg/kg/day, about 5 to 10 mg/kg/day, about 5 to 7.5 mg/kg/day) in mice, adjusted as appropriate to according to a patient's bodyweight and other considerations.

In regard to Ac-IGLHDPSHGTLPAGS, in certain embodiments, the compound is administered at a daily dosage in the range of about 0.1 mg to about 400 mg (e.g., about 0.1 mg to about 1 mg, about 1 mg to about 5 mg, about 5 mg to about 50 mg, about 50 mg to about 400 mg) for a time period of about 1 to about 14 days (e.g., about 1 to about 7 days, about 7 to about 14 days). In certain embodiments, the compound is administered at a daily dosage in the range of about 75 mg to about 300 mg for a time period of about 1 to about 7 (e.g., 1, 2, 3, 4, 5, 6 or 7) days. In certain embodiments, the compound is administered at a daily dosage in the range of about 100 mg to about 200 mg for a time period of about 1 to about 7 days (e.g., 1, 2, 3, 4, 5, 6 or 7).

In certain embodiments, the disclosed method further comprises administering to the subject a second therapeutic agent.

In certain embodiments, the second therapeutic agent is a small molecule compound. In certain embodiments, the second therapeutic agent is a small molecule antiviral agent. In certain embodiments, the second therapeutic agent is selected from remdesivir, dexamethasone, hydroxychloroquine, chloroquine, azithromycin, favipiravir, ribavirin and Lopinavir.

In certain embodiments, the second therapeutic agent is an antibody. In certain embodiments, the second therapeutic agent is selected from bamlanivimab (LY-CoV555), casirivimab and imdevimab (REGN-COV2), tocilizumab, sarilumab, baricitinib and levilimab.

In certain embodiments, the second therapeutic agent is convalescent plasma.

In certain embodiments, the second therapeutic agent is PAXLOVID™ (nirmatrelvir+ritonavir).

In certain embodiments, the second therapeutic agent is LAGEVRIO™ (molnupiravir).

In yet another aspect, the invention generally relates to a pharmaceutical composition comprising Ac-IGLHDPSHGTLPAGS, or a pharmaceutically acceptable form thereof, and a pharmaceutically acceptable excipient, carrier, or diluent, suitable for treating SARS-CoV-2 infection, or a related disease or condition.

In yet another aspect, the invention generally relates to a pharmaceutical composition comprising Ac-IGLHDPSHGTLPAGS, or a pharmaceutically acceptable form thereof, and a pharmaceutically acceptable excipient, carrier, or diluent, suitable for treating, reducing or preventing hypercytokinemia or cytokine storm or a disease or condition involving hypercytokinemia or cytokine storm.

In yet another aspect, the invention generally relates to a unit dosage form comprising a pharmaceutical composition disclosed herein.

In certain embodiments, the pharmaceutical composition is an aqueous formulation suitable for subcutaneous injection.

In certain embodiments, the aqueous formulation is stable at a temperature between about 2° C. to about 8° C. for at least 48 months.

In yet another aspect, the invention generally relates to use of Ac-IGLHDPSHGTLPAGS, or a pharmaceutically acceptable form thereof, for treating SARS-CoV-2 infection, or a related disease or condition.

In yet another aspect, the invention generally relates to use of Ac-IGLHDPSHGTLPAGS, or a pharmaceutically acceptable form thereof, for modulating the expression of Toll-like receptor 4 (TLR4) protein.

In yet another aspect, the invention generally relates to use of Ac-IGLHDPSHGTLPAGS, or a pharmaceutically acceptable form thereof, for reducing pro-inflammatory cytokines, or for treating, reducing or preventing hypercytokinemia or cytokine storm or a disease or condition involving hypercytokinemia.

In yet another aspect, the invention generally relates to use of Ac-IGLHDPSHGTLPAGS, or a pharmaceutically acceptable form thereof, for the manufacture of a medicament for preventing or treating post-COVID conditions, or a related disease or condition.

### Examples

The following examples are meant to be illustrative of the practice of the invention and not limiting in any way.

### Effects of Ac-IGLHDPSHGTLPAGS (API) on Survival Rate in LPS-Induced Mice Model

Severe systemic inflammation was induced in C57BL/6N mice with lipopolysaccharide (LPS) treatment, peritoneal contamination and infection (PCI), or cecal ligation and puncture (CLP). While LPS treatment elicited a stronger response than the others. The LPS and PCI models induced rapid onset of inflammation, including an early increase in serum pro-inflammatory cytokines and oxidative stress in organ tissues, a rapid decrease in blood glucose values and biotransformation capacity, immune cell infiltration from the circulation into the liver and spleen, and apoptosis in the spleen. LPS administration exhibited the strongest inflammatory effects of all of the models tested. The LPS model of systemic inflammation revealed to be most suitable when being interested in the impact of new therapies for acute inflammation. Liver function is affected in the three models of systemic inflammation.

### Materials, Instruments and Methods

Test article: Ac-IGLHDPSHGTLPAGS (Shenzhen Hightide Biopharmaceutical Ltd.)
Positive control: Dexamethasone (DEX) (Sangon Biotech (Shanghai) Co., Ltd.)
Vehicle control: 0.9% NaCl injection (Guizhou Kelun Pharmaceutical Co., Ltd.)
Lipopolysaccharide (LPS) (Sigma)

### Animals

Male SPF C57BL/6J mice (6-8 weeks old with body weight about 18-20 g) used in the study were purchased from Shanghai SLAC Laboratory Animal Co., Ltd. All the care and use of animals followed 3R principles and relevant regulations.

### Model Establishment and API Administration

One hundred (100) male C57BL/6J mice (18-20 g) were used for the study. After acclimated for 7 days, the inflammation mice model, which is characterized by a severe acute systemic inflammation response was established by a single tail vein injection of LPS (30 mg/kg).

The model animals were randomly divided into 7 groups: vehicle control group (G0), test group 1 (G1)˜test group 5 (G5) and positive control group (G6), of which there were 15 mice/group in G0˜G5 and 10 mice/group in G6. Day 1 was defined as the day on which tail vein injection of LPS (30 mg/kg), treatment start from the same day of the model establishment (Day 1) through Day 5 for consecutive 5 days (120 h after LPS induction). The detailed study design is summarized in Table 1 below. Except for the first dose on Day1 which were set at 5 min before LPS injection, 5 min after LPS injection, 1 hour before LPS injection and 1 hour after LPS injection respectively (Detailed information see the administration schedule in table 1), each groups have the same time schedules for administration (8 h after LPS injection for the second dose and bid with once in the morning, once in the evening for the following days). Vehicle and API were administered by back subcutaneous injection, DEX was administered by gavage.

TABLE 1
Study Design

| Group | | | Dosage | Dosage | | Time schedule of |
|---|---|---|---|---|---|---|
| ID | No./group | Treatment | (mg/kg/dosing) | (mg/kg/day) | Routes | administration |
| G0 | 15 | 0.9% NaCl | — | — | sc | Day 1: 5 min and 8 h |
| Vehicle | | injection | | | | after LPS injection |
| control | | | | | | Day 2-5: bid, once in |
| group | | | | | | the morning and once |
| | | | | | | in the evening |
| G1 | 15 | API | 1.25 | 2.5 | sc | Day 1: 5 min and 8 h |
| test | | | | | | after LPS injection |
| group 1 | | | | | | Day 2-5: bid, once in |
| | | | | | | the morning and once |
| | | | | | | in the evening |
| G2 | 15 | API | 3.75 | 7.5 | sc | Day 1: 5 min and 8 h |
| test | | | | | | after LPS injection |
| group 2 | | | | | | Day 2-5: bid, once in |
| | | | | | | the morning and once |
| | | | | | | in the evening: |
| G3 | 15 | API | 12.5 | 25 | sc | Day 1: 5 min and 8 h |
| test | | | | | | after LPS injection |
| group 3 | | | | | | Day 2-5: bid, once in |
| | | | | | | the morning and once |
| | | | | | | in the evening |
| G4 | 15 | API | 1.25 | 2.5 | sc | Day 1: 5 min before |
| test | | | | | | and 8 h after LPS |
| group 4 | | | | | | injection |
| | | | | | | Day 2-5: bid, once in |
| | | | | | | the morning and once |
| | | | | | | in the evening |
| G5 | 15 | API | 1.25 | 2.5 | sc | Day 1: 1 h and 8 h |
| test | | | | | | after LPS injection |
| group 5 | | | | | | Day 2-5: bid, once in |
| | | | | | | the morning and once |
| | | | | | | in the evening |
| G6 | 10 | DEX | 2 | 4 | ig | Day 1: 1 h before and |
| Positive | | | | | | 8 h after LPS injection |
| control | | | | | | Day 2-5: bid, once in |
| group | | | | | | the morning and once |
| | | | | | | in the evening |

### Results

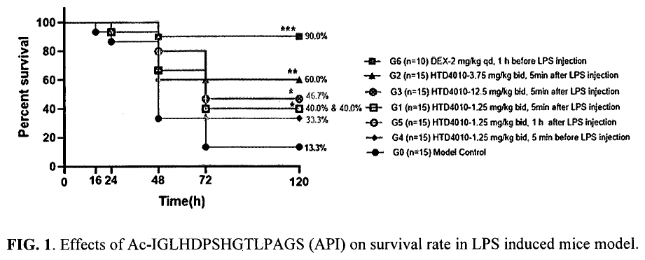

Graphpad 8.0 software was used for data analysis. Log-rank (Mantel-Cox) test was used for statistical analysis of the survival rate between each treatment group and vehicle control group. Results are provided in Table 2 and FIG. 1.

Tremors, lethargy, diarrhea and mental depression were generally observed 0-24 h after LPS injection for the mice from each group. Death was occurred 16 h after LPS injection for vehicle group and 24 h after LPS injection for test groups. Until 72 h after LPS injection, there were 13 mice in total died in vehicle group, 1 mouse died in positive control group, and 9, 6, 8, 11, 9 mice died in G1˜G5 groups respectively. After 72 h, no more mice died in any group till the end of the observation at 120 h.

Table 2 summarizes the survival rate of each group. As compared with the vehicle control group, the survival rate of G2, G3, G5 and G6 groups were significantly increased (P<0.05, P<0.01). The survival rate of G1 and G4 groups were not increased significantly (P>0.05), while the survival rate of G1 and G4 groups showed a trend of numerical improvement.

TABLE 2
Effects of API on the survival rate in LPS-induced mice model

| Group | 8 h | 16 h | 24 h | 48 h | 72 h | 120 h |
|---|---|---|---|---|---|---|
| G0 | 100.0% | 93.3% | 86.7% | 33.3% | 13.3% | 13.3% |
| Vehicle | | | | | | |
| control | | | | | | |
| group | | | | | | |
| G1 | 100.0% | 100.0% | 93.3% | 66.7% | 40.0% | 40.0% |
| Test | | | | | | |
| group 1 | | | | | | |
| G2 | 100.0% | 100.0% | 93.3% | 80.0% | 60.0% | 60.0%** |
| Test | | | | | | |
| group 2 | | | | | | |
| G3 | 100.0% | 100.0% | 93.3% | 66.7% | 46.7% | 46.7%* |
| Test | | | | | | |
| group 3 | | | | | | |
| G4 | 100.0% | 100.0% | 93.3% | 60.0% | 33.3% | 33.3% |
| Test | | | | | | |
| group 4 | | | | | | |
| G5 | 100.0% | 100.0% | 100.0% | 80.0% | 40.0% | 40.0%* |
| Test | | | | | | |
| group 5 | | | | | | |
| G6 | 100.0% | 100.0% | 100.0% | 90.0% | 90.0% | 90.0%** |
| Positive | | | | | | |
| control | | | | | | |
| group | | | | | | |

Note:
compared with vehicle control group,
*P < 0.05,
**P < 0.01,
***P < 0.001.

Results showed that API improved the survival rate of LPS-induced mice mortality, whereas Group3 achieved the most profound beneficial effects with the survival rate increased to 60% (FIG. 1).

Clinical Study of Ac-IGLHDPSHGTLPAGS (API) in Severe COVID-19 Patients with Elevated Inflammatory Factors

A protocol synopsis is provided in Table 3.

TABLE 3
Protocol Synopsis

| | |
|---|---|
| Drug under study | Active Pharmaceutical Ingredient (“API”): Ac-IGLHDPSHGTLPAGS |
| | (Acetyl-L-isoleucyl-glycyl-L-leucyl-L-histidinyl-L-aspartyl-L-prolyl-L- |
| | serinyl-L-histindinyl-glycyl-threonyl-L-leucyl-L-prolinyl-L-alanyl-glycyl- |
| | L-serine) |
| Placebo | Consistent appearance and weight with active drug |
| Route of | Subcutaneous injection |
| administration | |
| Intended Indication | Severe COVID-19 with Elevated Inflammatory Factors |
| Subjects | Patients with severe COVID-19 and elevated inflammatory factors |
| Number of subjects | ~75 |
| Study objectives | Primary objective |
| | To evaluate the effectiveness of API in the treatment of severe COVID-19 |
| | patients with elevated inflammatory factors |
| | Secondary objective |
| | To evaluate the safety of API in the treatment of severe COVID-19 patients |
| | with elevated inflammatory factors |
| Determination of | According to clinical practice, the proportion of severe COVID-19 patients |
| sample size | with elevated inflammatory factors whose IL-6 is reduced to normal within 7 |
| | days after treatment with placebo (combined with conventional treatment) is |
| | 40% (π1). It is assumed that API treatment (50 mg BID or 100 mg BID) can |
| | increase this proportion to 80% (π2), with the efficacy difference of 40%; set |
| | α = 0.05, β = 0.20, f (α, β) = 7.9 obtained from the reference table. The |
| | proportion of placebo group is planned to be 1:1 for each API dose group, |
| | and the estimated sample size is 20 subjects for each API dose group and 20 |
| | subjects for placebo group according to the formula n = [π1 (1 − π1) + π2 (1 − |
| | π2)]/(π2 − π1) 2 × f (α, β). Therefore, there are 20 patients in API 50 mg BID |
| | group, 20 patients in 100 mg BID group and 20 patients in placebo group. |
| | Considering a 20% dropout rate, a total of 75 subjects are planned to be |
| | enrolled. |
| Study design | This is an investigator-initiated, single-center, randomized, double-blind, |
| | placebo-controlled clinical trial to evaluate the efficacy and safety of API in |
| | 75 severe COVID-19 patients with elevated inflammatory factors. |
| | The study will be conducted in three cohorts of a total of 75 severe COVID- |
| | 19 patients with elevated inflammatory factors. Subjects who pass screening |
| | and sign informed consent will be randomized in a 1:1:1 ratio to Cohort 1 |
| | (API 50 mg twice daily, subcutaneous injection), Cohort 2 (API 100 mg twice |
| | daily, subcutaneous injection), and Cohort 3 (twice daily, subcutaneous |
| | injection of an equal amount of placebo), with 25 subjects in each cohort. |
| | During the study, all subjects will receive the standard of care recommended |
| | by the current COVID-19 guidelines (subject to the latest guidelines of the |
| | National Health Commission, with special reminders: glucocorticoids may be |
| | used during the study, but the guideline recommendations should be strictly |
| | followed). |
| | The subjects included in the study are subcutaneously injected with the |
| | study drug every 12 hours for 7 consecutive days starting on Day 1, during |
| | which blood samples for inflammatory parameters are collected and they are |
| | followed up to Day 14 to assess the efficacy and safety. |
| Study process and | This study is divided into screening period (Days −28~−2), baseline period |
| data acquisition | (Day −1), repeated dose period (Day 1-7) and follow-up period (Day 8-14). |
| | Eligible subjects are admitted to the study ward on the day before the |
| | anticipated dosing (Day −1), and receive subcutaneous injection of the study |
| | drug once every 12 hours (i.e., twice daily) for 7 consecutive days on Days |
| | 1-7, during which blood samples for inflammatory parameters are collected. |
| | Subjects will continue to be followed up for 14 days after randomization (Day |
| | 14) so as to monitor laboratory safety and efficacy measures. For the subjects |
| | with clinically significant AEs and abnormal laboratory safety indicators, the |
| | follow-up period should be extended until the events return to normal, |
| | baseline or reach a level that is considered stable by the investigator. |
| | The investigator will monitor the safety of the subjects by monitoring vital |
| | signs, physical examination, ECG and laboratory safety tests, determine |
| | AEs/SAEs according to the clinical significance of abnormal values, provide |
| | appropriate medical care to the subjects, and record or report according to the |
| | study protocol and regulatory requirements. |
| Statistical analysis | Efficacy analysis: |
| | Efficacy analyses are mainly based on the Full Analysis Set (FAS), while |
| | efficacy analyses are also carried out based on the Per Protocol Set (PPS). |
| | The log-rank test is used to compare the two groups of the primary endpoint |
| | of the study, and the hazard ratio (HR) and its 95% confidence interval of the |
| | primary endpoint are calculated by univariate Cox regression. An intent-to- |
| | treat analysis strategy is used for the primary endpoint. For the secondary |
| | efficacy endpoint, time to discharge, the Wilcoxon rank sum test is used to |
| | find the intergroup differences on a statistical basis. For other secondary |
| | efficacy endpoints and exploratory endpoints, the corresponding statistical |
| | methods are used based on the type of variables. For quantitative measures, |
| | the t test or Wilcoxon rank sum test (when t test is not applicable) is used, and |
| | for qualitative measures, chi-square test or accurate probability method (when |
| | chi-square test is not applicable) is used. Wilcoxon rank sum test is used for |
| | ranked data, and grade OR is calculated using grade logistic regression. |
| | Safety analysis: |
| | Safety is analyzed based on the Safety Set (SS). Adverse events are coded |
| | using Medical Dictionary for Regulatory Activities (MedDRA) terms (the |
| | most current version at the time of analysis), and descriptive statistics are |
| | provided by system organ (SOC)/preferred term (PT). The overall incidence |
| | of adverse events, adverse reactions, treatment-emergent adverse events, |
| | study drug-related adverse events, significant adverse events, serious adverse |
| | events, and the incidence by SOC/PT will be calculated. The number of |
| | patients with adverse events and adverse reactions during treatment and the |
| | number of episodes will be summarized by SOC and severity. Adverse |
| | events, adverse reactions, treatment-emergent adverse events, adverse events |
| | related to the study drug, significant adverse events and serious adverse |
| | events should be listed in detail. |
| | For laboratory tests, shift cross-tabulations of normal/abnormal results |
| | before and after administration are used to illustrate the changes in results |
| | before and after administration. Laboratory test results will be tabulated in |
| | detail. |

### Clinical Indications, Dosage and Route of Administration

The studied product is intended for the treatment of patients with severe COVID-19 and elevated inflammatory factors. Administration is by subcutaneous injection. The clinically studied dosage is not to exceed 300 mg/day and not more than 7 consecutive days.

From the perspective of clinical application, considering to the intended clinical indications, the majority of patients with COVID-19 and the immune response caused by cytokine storm are patients with acute and severe infections, oral administration is difficult. In addition, rapid absorption of drugs is required to control the progress of the disease. At the same time, small-volume injection also provides convenience for clinical application, which represents a simpler and quicker mode than powder for injection requiring redissolution.

API is a 15-amino acid peptide. Oral administration may degrade the peptide and fail to achieve the expected effects under existing technology. Subcutaneous injection is believed to be more favorable. Meanwhile, the results of stability study showed that the API has good stability and the injection can be safely stored at refrigeration temperature (2-8° C.).

### Preclinical Safety Data of API

A series of preclinical studies on pharmacology and pharmacokinetics of API were completed in accordance with ICH guidelines (The International Council for Harmonization of Technical Requirements for Pharmaceuticals for Human Use), and various preclinical safety evaluations including safety pharmacology, repeated-dose toxicity, genotoxicity test, in vitro hemolysis and in vivo and in vitro allergy tests were completed. No safety risk has been observed in connection with API at the maximum dose administered in each study. The no-observed-adverse-effect level (NOAEL) in each trial was the highest dose studied in each study, while the doses planned to be used for clinical trials are 150 mg/day and 300 mg/day, with a safety window of 9.7˜65.0 times.

A brief summary of the preclinical safety evaluations of API is provided in Table 4.

TABLE 4
Summary of Pre-clinical Safety Studies on API

| | | | | | |
|---|---|---|---|---|---|
| Type of | | | Dose | | Safety window |
| Testing | ROA | Species | (mg/kg) | Key Results | (Animal/human) |

Safety Pharmacology

| | | | | | |
|---|---|---|---|---|---|
| Respiratory | SC | SD | 0, 25, 125, 600 | NOAEL: 600 mg/kg | 38.7 |
| system | | (Sprague | | There was no obvious Respiratory | 19.4 |
| | | Dawley) | | System adverse effect in SD rats for | |
| | | rats | | the test article in all tested dosages. | |
| Cardiovascular | SC | Cynomolgus | 0, 15, 50, 150 | NOAEL: 150 mg/kg | 19.4 |
| System | | monkeys | | There was no obvious | 9.7 |
| | | | | Cardiovascular System adverse | |
| | | | | effect in SD rats for the test article in | |
| | | | | all tested dosages. | |
| Nervous system | SC | SD | 0, 25, 125, 600 | NOAEL: 600 mg/kg | 38.7 |
| | | (Sprague | | There was no obvious CNS adverse | 19.4 |
| | | Dawley) | | effect in SD rats for the test article in | |
| | | rats | | all tested dosages. | |

Repeated dose toxicity

| | | | | | |
|---|---|---|---|---|---|
| 14-day repeat | SC | C57BL/6 | 0, 60, 200, 600 | NOAEL: 600 mg/kg | 19.5 |
| dose phase + 7- | | mice | | No test article -related toxic findings | 9.8 |
| day recovery | | | | were observed at any dose level. | |
| phase | | | | | |
| 14-day repeat | SC | Cynomolgus | 0, 15, 50, 150 | NOAEL: 150 mg/kg | 19.4 |
| dose phase + 7- | | monkeys | | No test article related toxic findings | 9.7 |
| day recovery | | | | were observed at any dose level. | |
| phase | | | | | |

Genetic toxicology

| | | | | | |
|---|---|---|---|---|---|
| Ames assay | in | Salmonella | 0, 312.5, 625, | The test article is not mutagenic in | / |
| | vitro | typhimurium | 1250, 2500, | any of the test strains of Salmonella | |
| | | | 5000 μg/plate | typhimurium, TA97a, TA98, | |
| | | | | TA100, TA102 and TA1535. | |
| Chromosomal | in | CHL cells | 0, 62.5, 125, | The test article did not induce | / |
| Aberration | vitro | | 250, 500 | structural chromosome aberrations | |
| Assay, CHL | | | μg/mL | in CHL cells at concentration up to | |
| cells | | | | 500 μg/mL in either the presence or | |
| | | | | absence of S9 activation. | |
| Mouse | SC | ICRmice | 0, 500, 1000, | The test article did not show | 65.0 |
| micronucleus | | | 2000 | mutagenic activity in the mouse | 32.5 |
| | | | | micronucleus assay | |

In vitro hemolysis and in vivo and in vitro allergy test

| | | | | | |
|---|---|---|---|---|---|
| In vitro | in | Rabbit red | 26.7 mg/mL | The test article did not show | / |
| hemolysis | vitro | blood | | hemolysis and coagulation effects on | |
| | | cells | | rabbit red blood cells in vitro. | |
| Passive | SC | SD | 0, 5.4, 10.8 mg/ | The test article is negative for | / |
| cutaneous | | (Sprague | [symbol] | passive skin allergy test at any dose | |
| anaphylaxis | | Dawley) | | level. | |
| | | rats | | | |
| Active systemic | SC | Hartley | Allergenic | The test article is negative for active | / |
| anaphylaxis | | guinea pig | doses: 0, 9.3, | allergy at any dose level. | |
| | | | 18.6 mg/animal | | |
| | | | Challenge | | |
| | | | doses: 0, 18.6, | | |
| | | | 37.2 mg/animal | | |

### Clinical Study of API

A phase 1 clinical study with API in healthy subjects was completed. This trial was a randomized, double-blind, placebo-controlled trial on healthy subjects by one single administration, involving a total of four dose groups, 8 subjects in each group (6 of them were given API injection and 2 of them were given placebo), subcutaneous injection, at doses of 50, 100, 200 and 300 mg/day, respectively. The study results showed that after subcutaneous injection of API in healthy subjects, it was rapidly absorbed and eliminated, with a median Tₘₐₓ of 0.5 hours. By 4˜8 hours after administration, the plasma concentration decreased to the quantitative limit, about 2.5 ng/ml. The exposure and peak concentration were directly proportional to the dose. The pharmacokinetic parameters of API after subcutaneous injection are briefly summarized in Table 5; at 300 mg/day, the tolerability was good, no serious adverse reactions were found at each dose. All adverse reactions were very mild, the most common types were local redness and swelling at the injection site, and had improved or completely recovered at the end of the trial. No individual withdrew from the clinical trial for dosing reasons. The safety profile is summarized in Table 6. As can be seen above, API has excellent safety and tolerability in humans.

TABLE 5
Summary of pharmacokinetic parameters after single
subcutaneous injection in healthy adults

| | API dose (subcutaneous) | | | |
|---|---|---|---|---|
| PK Parameter | 50 mg | 100 mg | 200 mg | 300 mg |
| AUCₗₐₛₜ | 213 ± 27.6 | 497 ± 51.6 | 835 ± 89.8 | 1340 ± 143 |
| (ng · h/mL) | (n = 6) | (n = 6) | (n = 6) | (n = 6) |
| AUC_{inf} | 278 ± 32.7 | 537 ± 71.8 | 992 ± 113 | 1450 ± 199 |
| (ng · h/mL) | (n = 3) | (n = 4) | (n = 4) | (n = 4) |
| Cₘₐₓ | 132 ± 23.3 | 289 ± 51.6 | 405 ± 39.4 | 699 ± 143 |
| (ng/mL) | (n = 6) | (n = 6) | (n = 6) | (n = 6) |
| Tₘₐₓ ^{a} | 0.50 (0.25, 1.00) | 0.50 (0.50, 0.50) | 0.50 (0.25, 1.00) | 0.50 (0.25, 2.00) |
| (h) | (n = 6) | (n = 6) | (n = 6) | (n = 6) |
| Lambda (λz) | 0.768 ± 0.0640 | 1.07 ± 0.232 | 0.669 ± 0.0901 | 0.774 ± 0.146 |
| (1/h) | (n = 3) | (n = 4) | (n = 4) | (n = 4) |
| t_{1/2} | 0.915 ± 0.0764 | 0.753 ± 0.167 | 1.09 ± 0.142 | 0.972 ± 0.134 |
| (h) | (n = 3) | (n = 4) | (n = 4) | (n = 4) |
| DN AUCₗₐₛₜ | 4.25 ± 0.551 | 4.97 ± 0.516 | 4.18 ± 0.449 | 4.47 ± 0.478 |
| (ng · h/mL) | (n = 6) | (n = 6) | (n = 6) | (n = 6) |
| DN AUC_{inf} | 5.56 ± 0.653 | 5.37 ± 0.718 | 4.96 ± 0.567 | 4.83 ± 0.663 |
| (ng · h/mL) | (n = 3) | (n = 3) | (n = 4) | (n = 4) |
| DN Cₘₐₓ | 2.64 ± 0.466 | 2.89 ± 0.516 | 2.03 ± 0.197 | 2.33 ± 0.475 |
| (ng/mL) | (n = 6) | (n = 6) | (n = 6) | (n = 6) |

Note:
AUC_{inf} = area under the plasma concentration-time curve from time zero extrapolated to infinity;
AUCₗₐₛₜ = area under the plasma concentration-time curve from time zero to the time of the last quantifiable concentration;
Cₘₐₓ = maximum plasma concentration;
DN = dose-normalized;
t_{1/2} = half-life;
Tₘₐₓ = time to maximum concentration
^{a} Tₘₐₓ is represented by median (min, max).
Mean ± standard error (n, number of subjects) for all parameters except Tₘₐₓ.

TABLE 6
Summary of safety profile after single subcutaneous
administration in healthy adults

| | | API dose (subcutaneous) | | | | |
|---|---|---|---|---|---|---|
| | | | | | | Overall, in |
| | | | | | | the dosing |
| | Placebo | 50 mg | 100 mg | 200 mg | 300 mg | groups |
| | N = 8 | N = 6 | N = 6 | N = 6 | N = 6 | N = 24 |
| Adverse events | n (%) | n (%) | n (%) | n (%) | n (%) | n (%) |
| All adverse | 7 (87.5) | 5 (83.3) | 6 (100) | 6 (100) | 6 (100) | 23 (95.8) |
| events | | | | | | |
| Minor | 7 (87.5) | 5 (83.3) | 6 (100) | 6 (100) | 6 (100) | 23 (95.8) |
| Moderate | 0 | 0 | 0 | 0 | 0 | 0 |
| Severe | 0 | 0 | 0 | 0 | 0 | 0 |
| Serious adverse | 0 | 0 | 0 | 0 | 0 | 0 |
| event | | | | | | |
| Gastrointestinal | 1 (12.5) | 0 | 0 | 0 | 0 | 0 |
| reactions | | | | | | |
| (abdominal | | | | | | |
| pain) | | | | | | |
| Injection site | 7 (87.5) | 5 (83.3) | 6 (100) | 6 (100) | 6 (100) | 23 (95.8) |
| reactions | | | | | | |
| Erythema | 7 (87.5) | 5 (83.3) | 6 (100) | 6 (100) | 6 (100) | 23 (95.8) |
| Edema | 0 | 0 | 4 (66.7) | 3 (50) | 2 (33.3) | 9 (37.5) |

During the formulation study of API, special attention was paid and various effective measures such as avoiding the use of irritating substances in the drug product and controlling the pH of the drug product to be that for neutral isotonic preparation, so as to reduce the irritation at the injection site. Those measures were effective in reducing the irritation at the injection site and were confirmed in the human clinical phase 1 study of API. No individual withdrew from the clinical trial for the reasons of administration.

Data gathered from the preclinical safety study of API was deemed sufficient. Various preclinical safety evaluations including safety pharmacology, repeated-dose toxicity, genotoxicity test, in vitro hemolysis, in vitro and in vivo allergy test were completed. In the safety evaluations, no safety risk of API was observed at the maximum dose in each study. In the clinical phase 1 human study, after subcutaneous injection of API in healthy subjects, it was rapidly absorbed and eliminated, the exposure and peak concentration were directly proportional to the dose, with good human pharmacokinetic properties. At 300 mg/day, it was well tolerated, no serious adverse reactions were found at each dose, all adverse reactions were very mild, and no individual withdrew from the clinical trial due to administration reasons. API has excellent human safety and tolerability. The three similar products of API showed good safety and tolerability in multiple repeated doses in patients with different diseases; the dose as high as 600 mg/day was used in the human for the product of INGAP with only one amino acid composition difference as compared with API. Safety and tolerability of INGAP were good over the course of 90 days of continuous administration, which support the safety and tolerability of API during repeated administration in humans.

### Drug Substance and Drug Product for Clinical Trials

The studied API drug product is a small volume injection of 100 mg/mL. API injection was prepared using a conventional injection preparation process. The process steps included: dissolution, pH adjustment, filtration, filling, labeling packaging and release. The excipients used are conventional pharmaceutical excipients, which are sodium acetate, sodium hydroxide, sodium chloride and water for injection. One non-GMP batch and one GMP batch of API solution for injection have been manufactured. The product quality control standard is established with reference to the relevant provisions and requirements of ICHQ6A, in combination with its process and product characteristics; the quality control standard covers appearance, pH value, filling volume, osmotic pressure, insoluble particles, identity, content, related substances, sterility and bacterial endotoxin. The quality control analytical method is established based on the pharmacopoeia method, or developed by the sponsor. All the methods developed by the enterprise are established in accordance with ICH guidelines and the necessary methodological validation is completed, which can ensure the stability and reliability of the method.

The stability results of the existing GMP batches showed that all test measures of API injection met the quality control criteria after 48 months under the long-term storage condition of 2-8° C.

A protocol synopsis of the clinical study is provided in Table 3. It is noted that the clinical trial protocol disclosed herein may be modified, for example, for regulatory compliance and in consultation with regulatory authorities.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for treating SARS-CoV-2 infection, or a related disease or condition, comprising administering to a subject in need thereof a pharmaceutical composition comprising a compound that is an analogue of the active center of Reg3α.

2. The method of claim 1, wherein the compound is Ac-IGLHDPSHGTLPAGS, or a pharmaceutically acceptable form thereof.

3. The method according to claim 1, wherein the subject is in mild or moderate condition of SARS-CoV-2 infection, or a related disease or condition.

4. The method according to claim 1, wherein the subject is in severe or critical condition of SARS-CoV-2 infection, or a related disease or condition.

5. The method of claim 1, wherein the related disease or condition is pneumonia and/or lung injury.

6. The method of claim 1, wherein the related disease or condition is acute respiratory distress syndrome.

7. The method of claim 1, wherein the related disease or condition is systemic inflammatory response syndrome.

8. The method of claim 1, wherein the related disease or condition is cytokine storm.

9. The method of claim 1, wherein the related disease or condition is a cardiovascular condition.

10. The method of claim 1, wherein the related disease or condition is failure of one or more organs.

11. The method of claim 1, wherein the related disease or condition is lung failure.

12. The method of claim 1, wherein the related disease or condition is liver failure.

13. The method of claim 1, wherein the related disease or condition is a post-COVID condition.

14. A method of treating, reducing or preventing hypercytokinemia or cytokine storm or a disease or condition involving hypercytokinemia or cytokine storm, the method comprising administering to a subject in need thereof a pharmaceutical composition comprising a compound that is an analogue of the active center of Reg3α.

15. The method of claim 14, wherein the compound is Ac-IGLHDPSHGTLPAGS, or a pharmaceutically acceptable form thereof.

16. The method of claim 14, wherein the subject is suffering from an overproduction of immune cells and/or pro-inflammatory cytokines into the lungs of the subject.

17. The method of claim 16, wherein the pro-inflammatory cytokines comprise one or more of TNF-α, IL-1, IL-6, IL-12, IFN-α, IFN-β, IFN-γ, MCP-1, IL-8, IL-2, IL-7, IL-18, IL-17, CCL-2, IP-10, MCP-3 and GM-CSF.

18-35. (canceled)

36. The method of claim 1, further comprising administering to the subject a second therapeutic agent.

37-42. (canceled)

43. A pharmaceutical composition comprising Ac-IGLHDPSHGTLPAGS, or a pharmaceutically acceptable form thereof, and a pharmaceutically acceptable excipient, carrier, or diluent, suitable for treating SARS-CoV-2 infection, or a related disease or condition.

44. A unit dosage form comprising a pharmaceutical composition according to claim 43.

45. The unit dosage form of claim 44, wherein the pharmaceutical composition is an aqueous formulation suitable for subcutaneous injection, wherein the unit dosage form is stable at a temperature between about 2° C. to about 8° C. for at least 48 months.

46-51. (canceled)
